(19) United States
(12) Patent Application Publication
(10) Pub. No.: US 2024/0153089 A1
(43) Pub. Date: May 9, 2024

(54) SYSTEMS AND METHODS FOR PROCESSING REAL-TIME CARDIAC MRI IMAGES

(71) Applicant: Shanghai United Imaging Intelligence Co., Ltd., Shanghai (CN)

(72) Inventors: Xiao Chen, Lexington, MA (US); Zhang Chen, Brookline, MA (US); Terrence Chen, Lexington, MA (US); Shanhui Sun, Lexington, MA (US)

(73) Assignee: Shanghai United Imaging Intelligence Co., Ltd., Shanghai (CN)

(21) Appl. No.: 17/982,023

(22) Filed: Nov. 7, 2022

(51) Int. Cl. G06T 7/00; A61B 5/00; A61B 5/055; G06T 7/30

(52) U.S. Cl. CPC G06T 7/0016; A61B 5/0044; A61B 5/055; A61B 5/7267; G06T 7/30; G06T 2207/10088; G06T 2207/20081; G06T 2207/20212; G06T 2207/30048

(57) ABSTRACT

Real-time cardiac MRI images may be captured continuously across multiple cardiac phases and multiple slices. Machine learning-based techniques may be used to determine spatial (e.g., slices and/or views) and temporal (e.g., cardiac cycles and/or cardiac phases) properties of the cardiac images such that the images may be arranged into groups based on the spatial and temporal properties of the images and the requirements of a cardiac analysis task. Different groups of the cardiac MRI images may also be aligned with each other based on the timestamps of the images and/or by synthesizing additional images to fill in gaps.

### BACKGROUND

Cardiac magnetic resonance (CMR) is an important medical imaging tool for heart disease detection and treatment. Conventional CMR technologies often require patients to hold their breath during an imaging procedure so as to diminish the impact of respiratory motions, and an electrocardiogram (ECG) may also be needed in order to determine the cardiac phase of each CMR image and/or to combine data from multiple heart beats to form a single synthesized cardiac contraction cycle. In recent years, an alternative magnetic resonance imaging (MRI) technology called real-time CMR has been increasingly adopted for its faster and more flexible mode of operation. With real-time CMR, however, MRI signals (e.g., k-space data) may be acquired continuously (e.g., instead of always at the start of a specific cardiac phase or slice by slice) and, as such, determining the spatial and/or temporal alignment of the acquired images has posed a challenge.

### SUMMARY

Described herein are systems, methods, and instrumentalities associated with real-time cardiac MRI image processing. According to embodiments of the present disclosure, an apparatus capable of performing the real-time MRI image processing task may comprise at least one processor configured to obtain a plurality of medical images of a heart and determine, automatically, a slice and a cardiac phase associated with each of the plurality of medical images based on one or more machine-learned (ML) image recognition models. The plurality of medical images may be captured based on a real-time MRI technique and may span multiple cardiac phases and multiple slices of the heart. For example, the plurality of medical images may include a first medical image of the heart captured consecutively with a second medical image of the heart, where the first and second medical images may be associated with respective cardiac phases and slices, and where the first and second medical images may differ from each other with respect to at least one of the cardiac phases or the slices associated with the first and second medical images. Based at least on the automatically determined slice and cardiac phase information of each of the plurality of medical images, and a requirement of a cardiac analysis task, the at least processor may be further configured to select a first group of medical images from the plurality of medical images, and provide the first group of medical images for the cardiac analysis task.

In examples, the at least one processor of the apparatus may be further configured to determine, automatically, a view associated with the each of the plurality of medical images based on the one or more ML image recognition models, and select the first group of images further based on the view associated with the each of the plurality of medical images. Such a view may include, for example, a short-axis view, a 2-chamber long-axis view, a 3-chamber long-axis view, or a 4-chamber long-axis view of the heart.

In examples, the at least one processor of the apparatus may be further configured to select a second group of medical images from the plurality of medical images based at least on the requirement of the cardiac analysis task and the slice and cardiac phase associated with the each of plurality of medical images. The second group of medical images may be associated with a different cardiac cycle than the first group of medical images described above, and the second group of medical images may be misaligned with the first group of medical images with respect to one or more time spots. In such cases, the at least one processor of the apparatus may be configured to generate one or more additional medical images of the heart for the second group of medical images and add the one or more additional medical images to the second group of medical images such that the second group of medical images may be aligned with the first group of medical images with respect to the one or more time spots. The one or more additional medical images may be determined, for example, based on respective timestamps of the medical images comprised in the first and second groups. The one or more additional medical images may be generated, for example, based on an interpolation technique or a machine-learned image synthesis model.

In examples, the at least one processor of the apparatus may be further configured to register a first medical image of the first group of medical images with a second medical image of the first group of medical images, where the registration may compensate for a respiratory motion associated with the first medical image or the second medical image. In examples, the at least one processor may be further configured to perform the cardiac analysis task described above based on the first group of medical images.

### BRIEF DESCRIPTION OF THE DRAWINGS

A more detailed understanding of the examples disclosed herein may be had from the following description, given by way of example in conjunction with the accompanying drawings.

FIG. 1 is simplified diagram illustrating an example of grouping real-time CMR images based on spatial and temporal properties of the images automatically determined using one or more machine-learned (ML) image recognition models, according to some embodiments described herein.

FIG. 2 is a simplified diagram illustrating an example of an artificial neural network that may be used to implement and/or learn an image classification model, according to some embodiments described herein.

FIG. 3 is simplified diagram illustrating an example of an artificial neural network that may be used to implement and/or learn a segmentation model, according to some embodiments described herein.

FIG. 4 is a simplified diagram illustrating an example of an artificial neural network that may be used to implement and/or learn an image registration model, according to some embodiments described herein.

FIG. 5 is a flow diagram illustrating an example method for training a neural network to perform one or more of the tasks described with respect to some embodiments provided herein.

FIG. 6 is a simplified block diagram illustrating an example system or apparatus for performing one or more of the tasks described with respect to some embodiments provided herein.

### DETAILED DESCRIPTION

The present disclosure is illustrated by way of example, and not by way of limitation, in the figures of the accompanying drawings. A detailed description of illustrative embodiments will now be described with reference to the various figures. Although this description provides a detailed example of possible implementations, it should be noted that the details are intended to be exemplary and in no way limit the scope of the application.

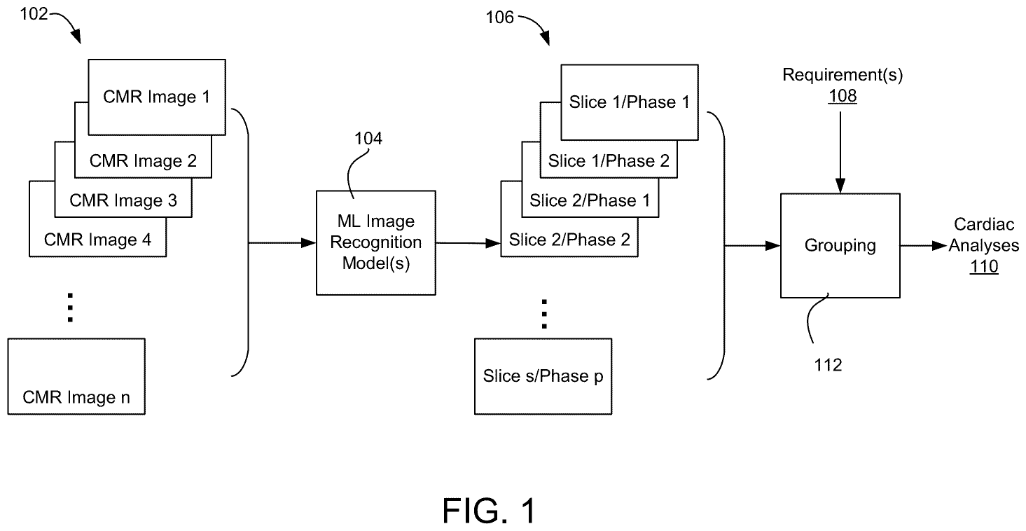

FIG. 1 illustrates an example of grouping real-time CMR images based on spatial and temporal properties of the images automatically determined using one or more machine-learned (ML) image recognition models. The CMR images 102 (e.g., which may be obtained in Digital Imaging and Communications in Medicine (DICOM) formats or other standard image formats such as JPEG) may be captured using real-time MRI technologies capable of continuously acquiring MRI signals (e.g., referred to herein as k-space data) and reconstructing them into the CMR images 102. With the real-time MRI technologies, image capturing may start at any point of a cardiac cycle and may end at any point of the cardiac cycle or a subsequent cardiac cycle. The image capturing operation may not be accompanied by physiological information (e.g., such as that provided by an ECG) or a requirement that the patient hold his or her breath while the images are being taken. Further, scanning of the patient's heart may move from one slice (e.g., corresponding to an orthogonal 2D plane along an axis of the heart) to another between consecutive time spots instead of staying at one slice location for an extended period at a time, as may be the case with a conventional or a retro-cine CMR procedure (e.g., a retro-cine CMR may capture a single slice at a time). Consequently, the CMR images 102 may span multiple cardiac phases (e.g., a systole or contraction phase, a diastole or relaxation phase, etc.) and multiple slices (e.g., along short and long axes of the heart), and may not be sequentially organized according to time and/or space positions (e.g., as may be the case with retro-cine CMR). For example, while CMR Image 1 may be captured at slice position 1 at time 1 (e.g., in the systole phase), a consecutively captured image (e.g., CMR Image 2) may be associated with a different slice (e.g., slice position 2) and/or a different cardiac phase (e.g., in the diastole phase). The respective views represented by consecutive images may also differ from each other. For instance, while CMR Image 1 may represent a short-axis view of the heart, CMR Image 2 may represent a 2-chamber long axis view of the heart, and CMR Image 3 may represent a 3-chamber long axis or a 4-chamber long axis view of the heart.

Machine learning techniques may be employed to automatically determine the temporal and spatial properties of the CMR images 102, and group the CMR images 102 based at least on these properties and the requirements of a specific clinical task (e.g., T1/T2 mapping, tissue characterization, medical abnormality detection, etc.) to be performed. The temporal properties may include, for example, the cardiac phase of each of the CMR images 102, the sequential order of the CMR images within a certain slice, etc., while the spatial properties may include, for example, the slice to which each of the CMR images 102 belongs, the view represented by each of the CMR images 102, etc. For example, as shown in FIG. 1, one or more ML image recognition models 104 may be trained and used to automatically determine, at 106, the slice, view, and/or cardiac phase associated with each of the CMR images 102. The automatically determined information may then be used, together with requirements 108 associated with one or more cardiac analysis tasks 110, to arrange the CMR images 102 into respective groups (e.g., at 112) to facilitate the cardiac analysis tasks 110. The terms “machine-learning,” “machine-learned,” “deep learning,” and “artificial intelligence” may be used interchangeably herein, and the terms “machine learning model,” “neural network,” and “deep neural network” may also be used interchangeably herein.

In examples, the one or more ML image recognition models 104 may include a first image classification model trained for separating the CMR images 102 into classes or categories corresponding to different slices or views of the heart. For instance, the first image classification model may be learned and/or implemented using a deep neural network (DNN) to classify each CRM image 102 into a category of views such as a short-axis view, a 2-chamber long-axis view, a 3-chamber long-axis view, a 4-chamber long-axis, etc. The CMR images 102 may be further classified to determine whether they belong to the same slice or different slices (e.g., a view may correspond to an angle at which an image is acquired, while a slice may correspond to a cut along that angle). For instance, the CMR images 102 may include a real-time CMR series comprising images {img_0_0_sax, img_1_0_sax, . . . , img_0_1_sax, img_1_1_sax, . . . , img_0_0_2ch, img_1_0_2ch, . . . , img_0_0_4ch, . . . }, where the first and second numerical values in the denotations may represent the time and slice locations at which the images are captured, respectively, and the last part of the denotations may represent the view (e.g., short-axis (sax), 2-chamber-long-axis (2ch), 3-chamber-long-axis (3ch), 4-chamber-long-axis (4ch), etc.) captured in each image. The DNN may be trained to learn features associated with the various slices and views through a training procedure, and subsequently determine, automatically, the slice and/or view associated with a given image based on the learned features. The determined slice and/or view information may then be used to arrange the CMR images 102 into different groups including, for example, a first group {img_0_0_sax, img_1_0_sax . . . } that may correspond to slice 0 of the short-axis view, a second group {img_0_1_sax, img_1_1_sax . . . } that may correspond to slice 1 of the short-axis view, a third group {img_0_0_2ch, img_1_0_2ch . . . } that may correspond to slice 0 of the 2-chamber long-axis view, a fourth group {img_0_0_4ch . . . } that may correspond to slice 0 of the 4-chamber long-axis view, etc. The DNN may take (e.g., only take) the CMR image 102 as inputs for determination the slice/view information of each image, or the DNN may take the CMR images 102 and other acquisition information (e.g., such as absolute acquisition times and/or locations included in a DICOM header) as inputs for the determination. In the latter case, the first image classification model may exploit the CMR images and acquisition information together or in a sequential order.

In examples, the one or more ML image recognition models 104 may include a second image classification model trained for determining the cardiac phase (e.g., within a cardiac cycle) associated with each CMR image 102. Such an ML model may also be learned and implemented using a DNN, which may be trained to learn features associated with various cardiac phases (e.g., end-of-diastole (ED), end-of-systole (ES), etc.) through a training procedure, and subsequently predict the cardiac phase depicted in a given image based on the learned features automatically. For instance, the CMR images 102 may include images {img_0_0_sax, img_0.3_0_sax, img_0.6_0_sax, img_0.9_0_sax, img_1.2_0_sax, img_1.5_0_sax, img_1.8_0_sax, img_2.1_0_sax . . . } spanning one or more cardiac phases or cycles, where the first number in the denotations may represent an absolute acquisition time (e.g., 0 may not necessarily correspond to the beginning of a cardiac cycle) of the image and the second number in the denotations may represent the slice to which the image may belong. Based on the features learned through training, the DNN may classify img_0.3_0_sax and img_1.5_0_sax as belonging to ED, and img_0.9_0_sax and img_2.1_0_sax as belonging to ES. The CMR images 102 may then be grouped into {img_0_0_sax}, {img_0.3_0_sax, img_0.6_0_sax, img_0.9_0_sax, img_1.2_0_sax}, and {img_1.5_0_sax, img_1.8_0_sax, img_2.1_0_sax} based on the determination of these key cardiac phases, where each group may correspond to a cardiac cycle and may include images starting from one ED and ending before the next ED. The rest of the images may be distributed into these groups based on the detected key cardiac phases, since those images may have been captured sequentially in time to reflect the continuous motion of the heart). In examples, a timestamp or time position relative to the first image in a group (e.g., which may be ED) may be assigned to each image in the group such that the images may be aligned with the images of another group, as will be described in greater detail below. The DNN may take (e.g., only take) the CMR image 102 as inputs for determining the cardiac phases of the images, or the DNN take the CMR images 102 and other acquisition information (e.g., such as absolute acquisition times included in a DICOM header) as inputs for the determination. In examples, additional ML-based processing may be conducted to facilitate the detection of the cardiac phases. For instance, the heart depicted in a CMR image 102 may be segmented using a segmentation network so as to obtain volumetric information of the heart (e.g., such as a left ventricle (LV) volume) as depicted in the image. The volumetric information may then be used to facilitate the determination of the cardiac phases, for example, since the ED and/or ES phases may have a strong association with the LV volume. The result of the segmentation operation may be re-used in a subsequent image analysis task (e.g., a post-analysis task) without incurring additional costs.

With the automatically determined time (e.g., cardiac phase) and space (e.g., slice/view) information of the CMR images 102, and the requirements 108 associated with the cardiac analysis tasks 110, the CMR images 102 may be grouped at 112 according to the requirements 108 and the automatically determined information. For example, with some cardiac analysis tasks, it may be desirable to examine the CMR images 102 grouped into different slices, where each slice may include images depicting a cardiac motion, while for other analysis tasks, it may be desirable to group the CMR images 102 into different cardiac phases, where each phase may include images spanning multiple slices and encompassing whole heart information.

In examples, all or a subset of the CMR images 102 may be arranged into groups that correspond to respective cardiac cycles, for example, upon tagging the images with automatically determined cardiac phase information and/or timestamp at 106. Two or more of these groups of images, however, may not be aligned with respect to time (e.g., for patients with heart diseases like premature ventricle contraction that may cause the timing of cardiac cycles to vary from one cycle to the next). For example, a first group corresponding to a first cardiac cycle may include 3 images with respective timestamps or time positions 1, 3, and 5 (e.g., relative to the first image in the first group), while a second group corresponding to a second cardiac cycle may include 5 images with respective timestamps or time positions of 1, 2, 3, 4, and 5 (e.g., relative to the first image in the second group). For clinical applications that may require CMR images to be time-aligned across different cardiac cycles, multiple CRM images in a group may be merged (e.g., into one image) or additional CMR images may be generated for a group (e.g., at the 2 and 4 time positions in the first group) such that the images in the group may be aligned with the images of another group (e.g., the second group mentioned above). The additional images may be generated using various interpolation techniques (e.g., linear interpolation techniques) based on existing images within a cardiac cycle and/or across different cardiac cycles (e.g., utilizing corresponding timestamps determined for the images associated with the different cardiac cycles). The additional images may also be generated using a neural network trained for image synthesis, e.g., by exploiting neighboring images that may be temporally related.

In examples, one or more of the CMR images 102 (e.g., belong to the same slice or cardiac phase) may be captured while the patient was engaged in a motion (e.g., a respiratory motion). The effects of such a motion on the CMR images (e.g., reflected through an in-plane translation) may be compensated using various image registration techniques including, for example, an image registration neural network. The motion compensation operation may be combined with motion related operations (e.g., such as motion estimation) in a post-analysis procedure such that the results of the motion compensation operation may be re-used during the post-analysis procedure without incurring additional computation or resource usage. In examples, a respiratory motion may not be removed via the motion compensation operation, and through-plane translation (e.g., cross-slice motion correction) may be accomplished by separating the CRM images into slices and grouping multiple slices together, e.g., using the image alignment techniques described herein.

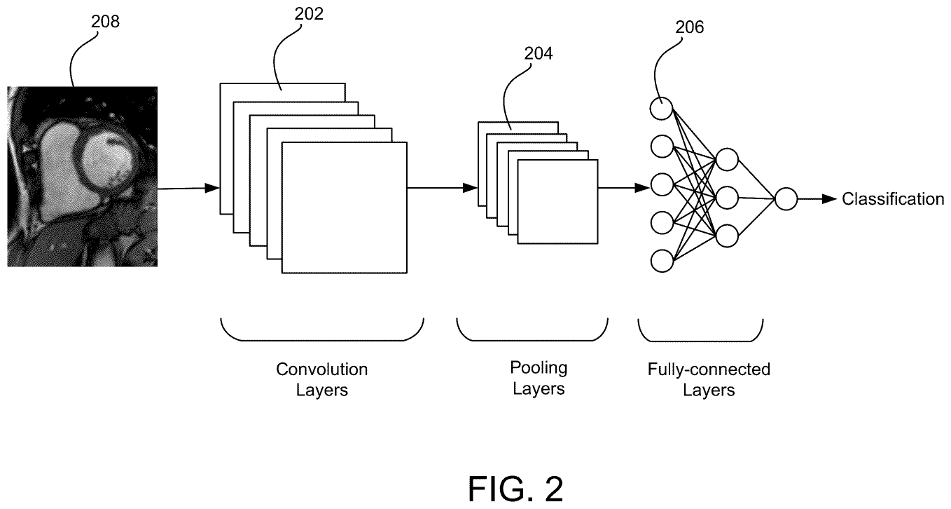

FIG. 2 illustrates an example of an artificial neural network (ANN) that may be used to implement and/or learn an image classification model such as the image classification model described herein (e.g., for classifying CMR images into different slices, views, and/or cardiac phases). The example is provided for illustration purposes and not to limit the scope of the disclosure. Those skilled in the art will understand that other machine-learning models such as a vision transformer may also be used to accomplish the goals described herein. As shown in FIG. 2, the ANN may be a convolutional neural network (CNN) that may include a plurality of layers such as one or more convolution layers 202, one or more pooling layers 204, and/or one or more fully connected layers 206. Each of the convolution layers 202 may include a plurality of convolution kernels or filters configured to extract features from an input image 208 (e.g., a cine image). The convolution operations may be followed by batch normalization and/or linear (or non-linear) activation, and the features extracted by the convolution layers may be down-sampled through the pooling layers and/or the fully connected layers to reduce the redundancy and/or dimension of the features, so as to obtain a representation of the down-sampled features (e.g., in the form of a feature vector or feature map). Based on the feature representation, a classification prediction may be made, for example, at an output of the ANN to indicate whether the input image 208 is a short-axis image (e.g., a short-axis slice), a long-axis image (e.g., a long-axis slice), a 2-chamber image, a 3-chamber image, a 4-chamber image, etc. The classification may be indicated with a label (e.g., “short-axis image,” “long-axis image,” “2-chamber image”, etc.), a numeric value (e.g., 1 corresponding to a short-axis image, 2 corresponding to a long-axis image, 3 corresponding to a 2-chamber image, etc.), and/or the like.

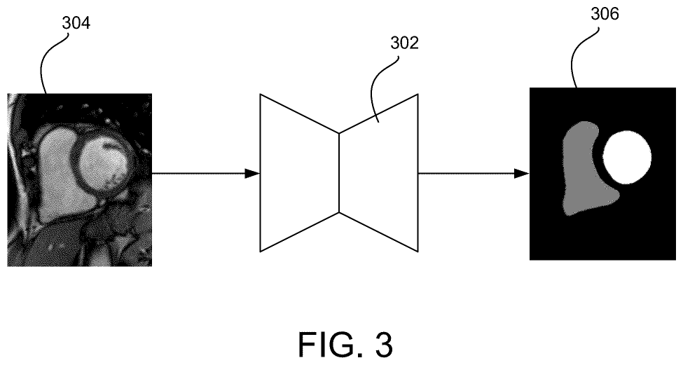

FIG. 3 illustrates an example of an artificial neural network (ANN) 302 that may be used to implement and/or learn an image segmentation model such as the image segmentation model described herein (e.g., for determine volumetric information of the heart based on a CMR image so as to determine a cardia phase associated with the CMR image). The ANN 302 may utilize an architecture that includes an encoder network and a decoder network (e.g., with one or more skip connections from the encoder network to the decoder network that are not explicitly shown in FIG. 3). The encoder network may be configured to receive an input image 304 such as a cine image, extract features from the input image, and generate a representation (e.g., a low-resolution or low-dimension representation) of the features at an output. The encoder network may be a convolutional neural network having multiple layers configured to extract and down-sample the features of the input image 304. For example, the encoder network may comprise one or more convolutional layers, one or more pooling layers, and/or one or more fully connected layers. Each of the convolutional layers may include a plurality of convolution kernels or filters configured to extract specific features from the input image. The convolution operation may be followed by batch normalization and/or non-linear activation, and the features extracted by the convolutional layers (e.g., in the form of one or more feature maps) may be down-sampled through the pooling layers and/or the fully connected layers to reduce the redundancy and/or dimension of the features. The feature representation produced by the encoder network may be in various forms including, for example, a feature map or a feature vector.

The decoder network of ANN 302 may be configured to receive the representation produced by the encoder network, decode the features of the input image 304 based on the representation, and generate a mask 306 (e.g., a pixel- or voxel-wise segmentation mask) for segmenting one or more objects (e.g., the LV and/or RV of a heart, the AHA heart segments, etc.) from the input image 302. The decoder network may also include a plurality of layers configured to perform up-sampling and/or transpose convolution (e.g., deconvolution) operations on the feature representation produced by the encoder network, and to recover spatial details of the input image 304. For instance, the decoder network may include one or more un-pooling layers and one or more convolutional layers. Through the un-pooling layers, the decoder network may up-sample the feature representation produced by the encoder network (e.g., based on pooled indices stored by the encoder network). The up-sampled representation may then be processed through the convolutional layers to produce one or more dense feature maps, before batch normalization is applied to the one or more dense feature maps to obtain a high dimensional representation of the input image 304. As described above, the output of the decoder network may include a segmentation mask for delineating one or more anatomical structures or regions from the input image 304. In examples, such a segmentation mask may correspond to a multi-class, pixel/voxel-wise probabilistic map in which pixels or voxels belonging to each of the multiple classes are assigned a high probability value indicating the classification of the pixels/voxels.

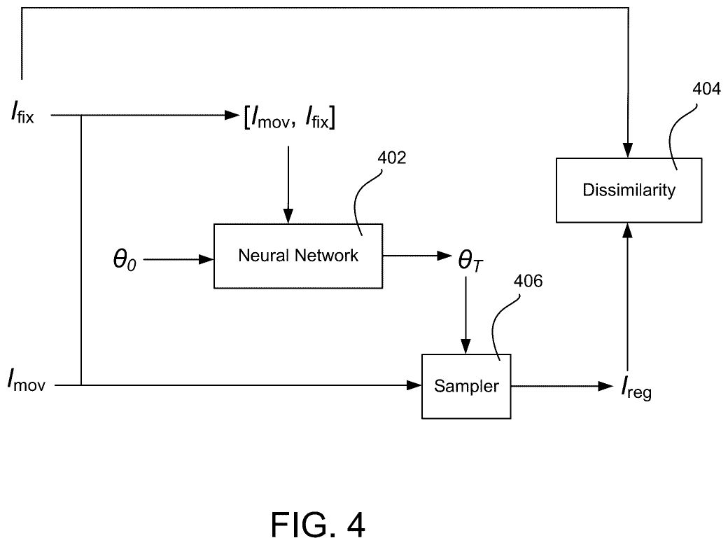

FIG. 4 illustrates an example of registering two CMR images, Iₘₒᵥ (e.g., a source CMR image) and I_{fix} (e.g., a target CMR image), using an artificial neural network (ANN) 402, to compensate for a motion (e.g., a respiratory motion) associated with the images. As shown, the neural network 402 may be configured to receive the images I_{fix} and Iₘₒᵥ (e.g., as inputs), transform the image Iₘₒᵥ from a moving image domain (e.g., associated with the image Iₘₒᵥ) to a fixed image domain (e.g., associated with the image I_{fix}), and generate an image I_{reg} (e.g., as a spatial transformed version of the image Iₘₒᵥ) that may resemble the image I_{fix} (e.g., with a minimized dissimilarity 404 between I_{fix} and I_{reg}). The neural network 402 may be trained to determine a plurality of transformation parameters OT for transforming the image Iₘₒᵥ into the image I_{reg}, as illustrated by the equation below:

I_{reg}=Iₘₒᵥ(θ(x)) (1)

where x may represent coordinates in the moving image domain, θ(x) may represent the mapping of x to the fixed image domain, and Iₘₒᵥ (θ(x)) may represent one or more grid sampling operations (e.g., using a sampler 406). θ may include parameters associated with an affine transformation model, which may allow for translation, rotation, scaling, and/or skew of the input image. θ may also include parameters associated with a deformable field (e.g., a dense deformation field), which may allow for deformation of the input image. For example, θ may include rigid parameters, B-spline control points, deformable parameters, and/or the like.

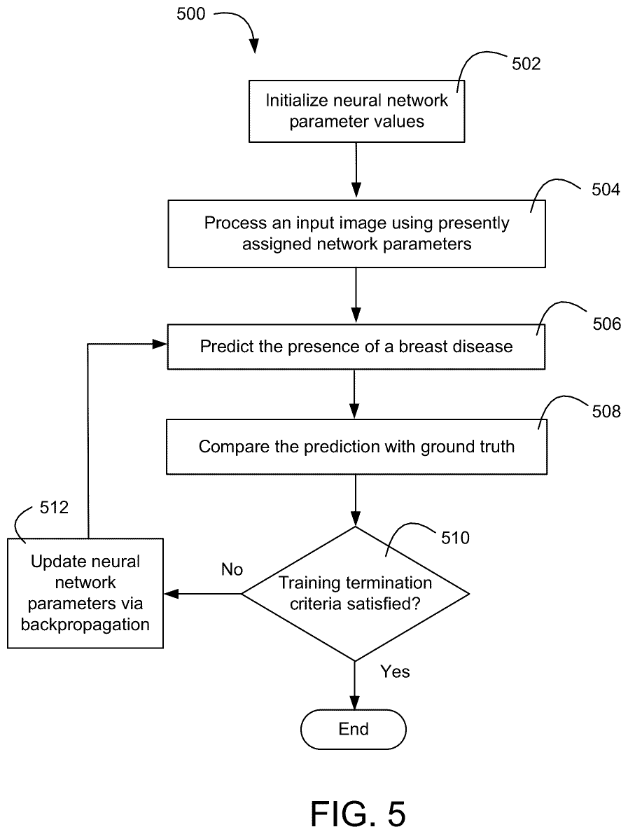

FIG. 5 shows a flow diagram illustrating an example process 500 for training a neural network (e.g., an ML model implemented by the neural network) to perform one or more of the tasks described herein. As shown, the training process 500 may include initializing the operating parameters of the neural network (e.g., weights associated with various layers of the neural network) at 502, for example, by sampling from a probability distribution or by copying the parameters of another neural network having a similar structure. The training process 500 may further include processing an input training image (e.g., a cine image or tissue characterization map) using presently assigned parameters of the neural network at 504, and making a prediction about a desired result (e.g., a classification label, a segmentation mask, a motion compensated image, etc.) at 506. The prediction result may be compared, at 508, to a ground truth, and a loss associated with the prediction may be determined based on the comparison and a loss function. The loss function employed for the training may be selected based on the specific task that the neural network is trained to do. For example, if the task involves a classification or segmentation of the input image, or minimizing the difference between an original image and a warped image (e.g., for image registration), a loss function based on a mean squared error between the prediction result and the ground truth may be used.

At 510, the loss calculated using one or more of the techniques described above may be used to determine whether one or more training termination criteria are satisfied. For example, the training termination criteria may be determined to be satisfied if the loss is below a threshold value or if the change in the loss between two training iterations falls below a threshold value. If the determination at 510 is that the termination criteria are satisfied, the training may end; otherwise, the presently assigned network parameters may be adjusted at 512, for example, by backpropagating a gradient descent of the loss function through the network before the training returns to 506.

For simplicity of explanation, the training steps are depicted and described herein with a specific order. It should be appreciated, however, that the training operations may occur in various orders, concurrently, and/or with other operations not presented or described herein. Furthermore, it should be noted that not all operations that may be included in the training method are depicted and described herein, and not all illustrated operations are required to be performed.

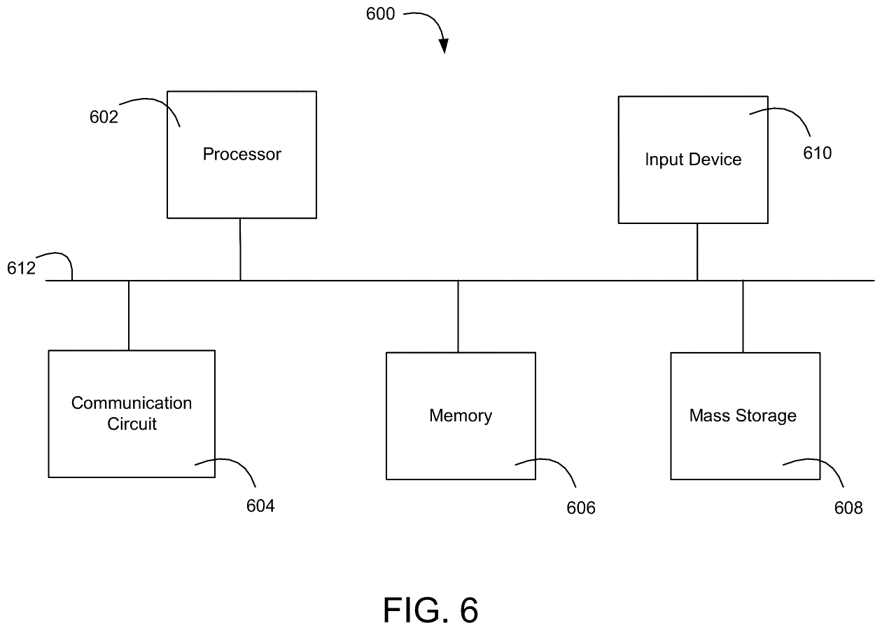

The systems, methods, and/or instrumentalities described herein may be implemented using one or more processors, one or more storage devices, and/or other suitable accessory devices such as display devices, communication devices, input/output devices, etc. FIG. 6 illustrates an example of an apparatus 600 that may be configured to perform the tasks described herein. As shown, apparatus 600 may include at least one processor (e.g., one or more processors) 602, which may be a central processing unit (CPU), a graphics processing unit (GPU), a microcontroller, a reduced instruction set computer (RISC) processor, application specific integrated circuits (ASICs), an application-specific instruction-set processor (ASIP), a physics processing unit (PPU), a digital signal processor (DSP), a field programmable gate array (FPGA), or any other circuit or processor capable of executing the functions described herein. Apparatus 600 may further include a communication circuit 604, a memory 606, a mass storage device 608, an input device 610, and/or a communication link 612 (e.g., a communication bus) over which the one or more components shown in the figure may exchange information.

Communication circuit 604 may be configured to transmit and receive information utilizing one or more communication protocols (e.g., TCP/IP) and one or more communication networks including a local area network (LAN), a wide area network (WAN), the Internet, a wireless data network (e.g., a Wi-Fi, 3G, 4G/LTE, or 5G network). Memory 606 may include a storage medium (e.g., a non-transitory storage medium) configured to store machine-readable instructions that, when executed, cause processor 602 to perform one or more of the functions described herein. Examples of the machine-readable medium may include volatile or non-volatile memory including but not limited to semiconductor memory (e.g., electrically programmable read-only memory (EPROM), electrically erasable programmable read-only memory (EEPROM)), flash memory, and/or the like. Mass storage device 608 may include one or more magnetic disks such as one or more internal hard disks, one or more removable disks, one or more magneto-optical disks, one or more CD-ROM or DVD-ROM disks, etc., on which instructions and/or data may be stored to facilitate the operation of processor 602. Input device 610 may include a keyboard, a mouse, a voice-controlled input device, a touch sensitive input device (e.g., a touch screen), and/or the like for receiving user inputs to apparatus 600.

It should be noted that apparatus 600 may operate as a standalone device or may be connected (e.g., networked or clustered) with other computation devices to perform the tasks described herein. And even though only one instance of each component is shown in FIG. 6, a skilled person in the art will understand that apparatus 600 may include multiple instances of one or more of the components shown in the figure.

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure. In addition, unless specifically stated otherwise, discussions utilizing terms such as “analyzing,” “determining,” “enabling,” “identifying,” “modifying” or the like, refer to the actions and processes of a computer system, or similar electronic computing device, that manipulates and transforms data represented as physical (e.g., electronic) quantities within the computer system's registers and memories into other data represented as physical quantities within the computer system memories or other such information storage, transmission or display devices.

It is to be understood that the above description is intended to be illustrative, and not restrictive. Many other implementations will be apparent to those of skill in the art upon reading and understanding the above description.

What is claimed is:

1. An apparatus, comprising:
at least one processor configured to:
obtain a plurality of medical images of a heart;
determine, based on one or more machine-learned (ML) image recognition models, a slice and a cardiac phase associated with each of the plurality of medical images;
select a first group of medical images from the plurality of medical images based at least on the slice and cardiac phase associated with each of the plurality of medical images and a requirement of a cardiac analysis task; and
provide the first group of medical images for performing the cardiac analysis task.

2. The apparatus of claim 1, wherein the plurality of medical images is captured based on a real-time magnetic resonance imaging (MRI) technique and spans multiple cardiac phases and multiple slices of the heart.

3. The apparatus of claim 2, wherein the plurality of medical images includes a first medical image of the heart captured consecutively with a second medical image of the heart, the first and second medical images being associated with respective cardiac phases and slices, and wherein the first and second medical images differ from each other with respect to at least one of the cardiac phases or the slices associated with the first and second medical images.

4. The apparatus of claim 1, wherein the at least one processor is further configured to determine, automatically, a view associated with each of the plurality of medical images based on the one or more ML image recognition models, and select the first group of medical images further based on the view associated with each of the plurality of medical images.

5. The apparatus of claim 4, wherein the view includes a short-axis view, a 2-chamber long-axis view, a 3-chamber long-axis view, or a 4-chamber long-axis view of the heart.

6. The apparatus of claim 1, wherein the first group of medical images is associated with a first cardiac cycle, and wherein the at least one processor is further configured to:
select a second group of medical images from the plurality of medical images based at least on the requirement of the cardiac analysis task and the slice and cardiac phase associated with each of plurality of medical images, wherein the second group of medical images is associated with a second cardiac cycle and is misaligned with the first group of medical images with respect to one or more time spots;
generate one or more additional medical images of the heart for the second group of medical images; and
add the one or more additional medical images to the second group of medical images such that the second group of medical images is aligned with the first group of medical images with respect to the one or more time spots.

7. The apparatus of claim 6, wherein the at least one processor is further configured to determine respective timestamps of the medical images comprised in the first group of medical images and the second group of medical images, and wherein the one or more additional medical images are generated for the second group of medical images based at least on the determined timestamps.

8. The apparatus of claim 6, wherein the one or more additional medical images are generated based on an interpolation technique or a machine-learned image synthesis model.

9. The apparatus of claim 1, wherein the at least one processor is further configured to register a first medical image of the first group of medical images with a second medical image of the first group of medical images, the registration compensating for a respiratory motion associated with the first medical image or the second medical image.

10. The apparatus of claim 1, wherein the at least one processor is further configured to perform the cardiac analysis task based on the first group of medical images.

11. A method of processing medical images, the method comprising:
obtaining a plurality of medical images of a heart;
determining, based on one or more machine-learned (ML) image recognition models, a slice and a cardiac phase associated with each of the plurality of medical images;
selecting a first group of medical images from the plurality of medical images based at least on the slice and cardiac phase associated with each of the plurality of medical images and a requirement of a cardiac analysis task; and
providing the first group of medical images for performing the cardiac analysis task.

12. The method of claim 11, wherein the plurality of medical images is captured based on a real-time magnetic resonance imaging (MRI) technique and spans multiple cardiac phases and multiple slices of the heart.

13. The method of claim 12, wherein the plurality of medical images includes a first medical image of the heart captured consecutively with a second medical image of the heart, the first and second medical images being associated with respective cardiac phases and slices, and wherein the first and second medical images differ from each other with respect to at least one of the cardiac phases or the slices associated with the first and second medical images.

14. The method of claim 11, further comprising determining, automatically, a view associated with each of the plurality of medical images based on the one or more ML image recognition models, and selecting the first group of medical images further based on the view associated with each of the plurality of medical images.

15. The method of claim 14, wherein the view includes a 2-chamber view, a 3-chamber view, or a 4-chamber view of the heart.

16. The method of claim 11, wherein the first group of medical images is associated with a first cardiac cycle, and wherein the method further comprises:
selecting a second group of medical images from the plurality of medical images based at least on the requirement of the cardiac analysis task and the slice and cardiac phase associated with each of plurality of medical images, wherein the second group of medical images is associated with a second cardiac cycle and is misaligned with the first group of medical images with respect to one or more time spots;
generating one or more additional medical images of the heart for the second group of medical images; and
adding the one or more additional medical images to the second group of medical images such that the second group of medical images is aligned with the first group of medical images with respect to the one or more time spots.

17. The method of claim 16, further comprising determining respective timestamps of the medical images comprised in the first group of medical images and the second group of medical images, wherein the one or more additional medical images are generated for the second group of medical images based at least on the determined timestamps.

18. The method of claim 16, wherein the one or more additional medical images are generated using an interpolation technique or a machine-learned image synthesis model.

19. The method of claim 11, further comprising registering a first medical image of the first group of medical images with a second medical image of the first group of medical images, wherein the registration compensates for a respiratory motion associated with the first medical image or the second medical image.

20. The method of claim 11, further comprising performing the cardiac analysis task based on the first group of medical images.
